(19) United States
(12) Patent Application Publication
(10) Pub. No.: US 2022/0072342 A1
(43) Pub. Date: Mar. 10, 2022

(54) NOSE BRIDGE STRIP WITH SCENT RELEASING MECHANISM AND MASK STRUCTURE HAVING THE SAME

(71) Applicant: Cheng-Lang TSAI, New Taipei City (TW)

(72) Inventor: Cheng-Lang TSAI, New Taipei City (TW)

(21) Appl. No.: 17/391,264

(22) Filed: Aug. 2, 2021

(30) Foreign Application Priority Data
Sep. 4, 2020 (TW) 109130367

(51) Int. Cl. A62B 9/06; A41D 13/11; A61M 15/00; A61L 9/012

(52) U.S. Cl. CPC A62B 9/06; A41D 13/11; A61L 2209/15; A61L 9/012; A61M 15/0001

(57) ABSTRACT

A nose bridge strip having a scent-releasing mechanism can be included in a mask structure and includes a main body and at least one scent-releasing element. The main body is in a shape of a strip and can be combined with the scent-releasing element. The scent-releasing element can produce scent molecules capable of reacting with olfactory cells in a nasal cavity such that bioelectric waves are generated and propagate to the brain. The mask structure includes the nose bridge strip and a mask body that can be arranged with the nose bridge strip. When the mask body is worn on a human face, the nose bridge strip can be pressed and deformed compliantly against the nose bridge so that a user wearing the mask structure can smell a pleasant scent while breathing and feel comfortable.

### CROSS-REFERENCE TO RELATED PATENT APPLICATION

This non-provisional application claims priority to and the benefit of, under 35 U.S.C. § 119(a), Taiwan Patent Application No. 109130367, filed in Taiwan on Sep. 4, 2020. The entire content of the above identified application is incorporated herein by reference.

### FIELD

The present disclosure is related to a nose bridge strip and a mask structure having the same, and more particularly to a nose bridge strip and a mask structure having the same that have a scent-releasing mechanism that enables a user wearing the mask structure to smell a pleasant scent while breathing.

### BACKGROUND

“PM2.5” refers to particulate matter with an aerodynamic diameter of 2.5 μm or less and is therefore also known as fine particles. These fine particles (hereinafter referred to as PM2.5) are produced not only by human behaviors (e.g., the exhaust of motor vehicles, tobacco smoke, and emissions from coal-fired power stations), but also by natural processes such as dust storms and volcanic eruptions. PM2.5 can be generally categorized as primary or secondary. Primary PM2.5 refers to particles that already fit the PM2.5 definition when emitted into the atmosphere, including for example suspended particles of sea salt, carbon particles emitted from motor vehicles, dust flying up from the road surface, and carbon particles of coal. Secondary PM2.5 (also referred to as secondary aerosols) refers to particles formed of even finer emissions by way of a physical reaction (e.g., condensation) or chemical reaction (e.g., photochemical reaction), including for example sulfates, nitrates, ammonium salts, and organic aerosols.

PM2.5 poses a serious threat to human health because the surface of PM2.5 may adsorb a large amount of toxic substances such as dioxin, polycyclic aromatic hydrocarbons, mercury, lead, and benzene, which in turn may pass through the barriers of the human respiratory system and go deep into the lungs along with PM2.5 due to the small particle sizes of PM2.5. Studies conducted by the World Health Organization (WHO) have shown that on a yearly global basis, about 3% and 5% respectively of cardiopulmonary diseases and lung cancer can be attributed to PM2.5, causing about 3.1 million deaths per year globally. In particular, PM2.5 containing an acidic aerosol such as a sulfate is highly hazardous. According to the United States National Air Pollution Control Administration, most acidic aerosols have particle sizes smaller than 2.5 μm, can be deposited in the lower respiratory tract and the alveoli through respiration, and may directly result in a reduction or impairment of the functions of the lungs and the respiratory tract and thus affect human health. Such acidic aerosols are detrimental to, and may raise the chronic disease morbidity of, those who are hypersensitive, such as the elderly, children, and patients with a respiratory disease.

In view of the above, some people choose to wear a personal protective device (e.g., a mask) to lower the risk of exposure to PM2.5. Furthermore, the recent pandemic of coronavirus disease 2019 (COVID-19) has turned mask wearing into an essential self-protective behavior. Wearing a mask for an extended period of time, however, has its side effects, including for example a painful sensation of pressure in the head, dizziness, nausea, and low spirits, all of which may lead to weariness. In addition, a mask may gradually develop an unpleasant smell as its user breathes in and out through the mask, coughs or sneezes in the mask, and puts the mask hack on after each meal. The issue to be addressed by the present disclosure is to improve the conventional mask structures so as to provide mask users with better user experience.

### SUMMARY

As a conventional mask structure is prone to cause displeasing odor at the inner side thereof after use, which not only causes suffocating discomfort to a user wearing the mask structure, but also makes the user to fear for embarrassment if someone else smells the odor when the mask structure is removed, based on years of rich practical experience, excelling research spirit, and longtime and labored research and experiment, the present disclosure provides a nose bridge strip with a scent releasing mechanism and a mask structure having the same to allow a user to smell a pleasant scent while breathing even after daylong mask wearing.

One aspect of the present disclosure is directed to a nose bridge strip having a scent releasing mechanism. The nose bridge strip includes a main body and at least one scent releasing element. The main body is in a shape of a strip. The main body can be fixed on or in a mask body at a location corresponding to a nose bridge on a human face when the mask body is worn on the human face, and can be pressed and deformed compliantly against the nose bridge. The at least one scent releasing element can be combined with the main body and produce scent molecules capable of reacting with olfactory cells in a nasal cavity.

Another aspect of the present disclosure is directed to a mask structure having a scent releasing mechanism. The mask structure includes a mask body, ear straps, a main body, and at least one scent releasing element. The mask body includes at least one air-permeable layer. The ear straps are provided respectively at the left and right sides of the mask body, and can be wrapped around left and right ears of a user and bring an inner-side surface of the mask body close to the face of the user. The main body is in a shape of a strip, and can be fixed on or in a mask body at a location corresponding to a nose bridge on the face when the mask body is worn on the face, and be pressed and deformed compliantly against the nose bridge. The at least one scent releasing element can be combined with the main body and produce scent molecules capable of reacting with olfactory cells in a nasal cavity, so that a user wearing the mask structure can smell a pleasant scent while breathing.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic diagram of a mask structure according to certain embodiments of the present disclosure.

FIG. 2 is a cross-sectional view of the mask structure according to certain embodiments of the present disclosure.

FIG. 3 is a cross-sectional view of a nose bridge strip according to certain embodiments of the present disclosure.

FIG. 4 is a cross-sectional view of a nose bridge strip according to certain embodiments of the present disclosure.

FIG. 5 is a perspective view of a nose bridge strip in which adjacent turns of a film are spaced apart from each other according to certain embodiments of the present disclosure.

FIG. 6 is a cross-sectional view of a nose bridge strip according to certain embodiments of the present disclosure.

FIG. 7 is a perspective view of a groove provided on a nose bridge strip according to certain embodiments of the present disclosure.

FIG. 8 is an exploded perspective view of a groove provided on a nose bridge strip according to certain embodiments of the present disclosure.

### DETAILED DESCRIPTION

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. The numbering terms such as “first”, “second” or “third”, etc. can be used to describe various components, parts or the like, and the directional terms such as “upper”, “tower”, “front”, “rear”, “right”, “left”, etc., can be used to describe the direction of the components, parts or the like, which are for distinguishing one component/part from another one, and distinguishing one direction of one component/part from another one only, and are not intended to, nor should be construed to impose any substantive limitations on the components, parts or the like or the directions thereof, or be relevant to the sequence in which the components/parts are to be assembled or disposed in practical application.

As used herein, the term “substantially” or “approximately” refers to, for example, a value, or an average of values, in an acceptable deviation range of a particular value recognized or decided by a person of ordinary skill in the art, taking into account any specific quantity of errors related to the measurement of the value that may resulted from limitations of a measurement system or device. For example, “substantially” may indicate that the value is within, for example, ±5%, ±3%, ±1%, ±0.5% or ±0.1%, or one or more standard deviations, of the particular value.

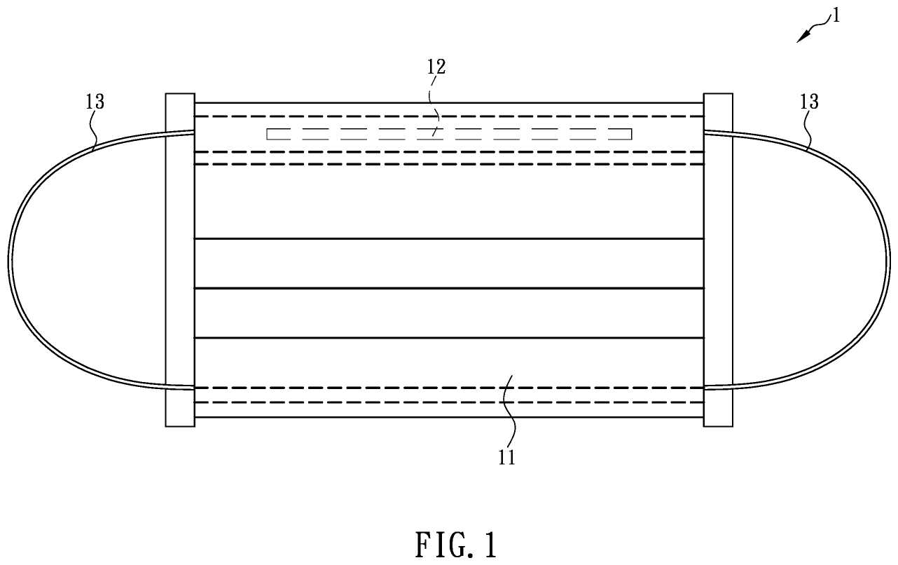

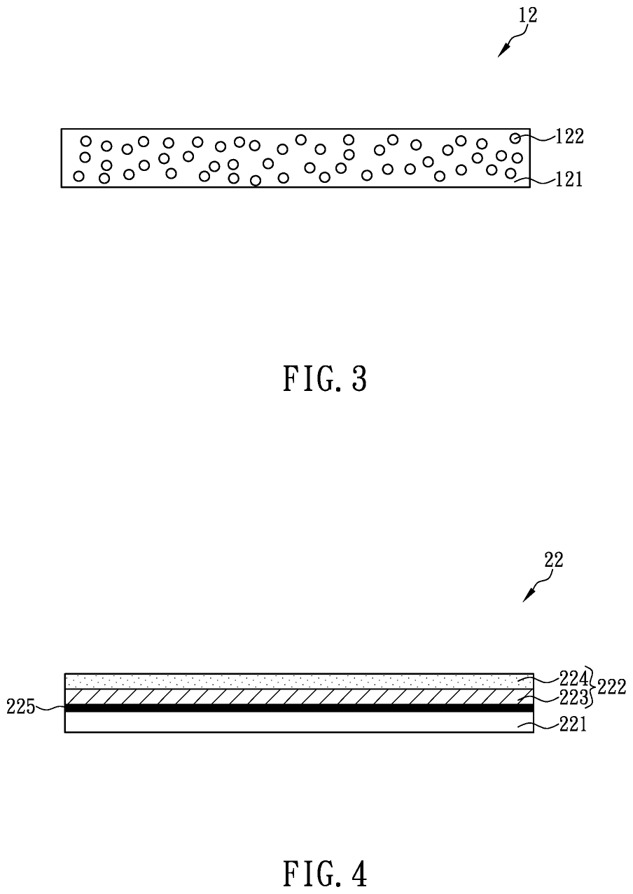

The present disclosure provides a nose bridge strip having a scent releasing mechanism and a mask structure having the nose bridge strip. Referring to FIG. 1 and FIG. 3, the mask structure 1 according to certain embodiments includes a mask body 11 and a nose bridge strip 12. The nose bridge strip 12 can be formed by combining a main body 121 with at least one scent releasing element 122. The nose bridge strip 12 can be provided on or in the mask body 11. The main body 121 can be in a shape of a strip. Each scent releasing element 122 can produce scent molecules, thereby providing the nose bridge strip 12 with a scent releasing mechanism, allowing one who is wearing the mask structure 1 to smell a pleasant scent while breathing. Besides, each of the left and right sides of the mask body 11 can be provided with an ear strap 13 to be wrapped around a user's left and/or right ear so that the inner-side surface of the mask body 11 is close to the user's face and covers the user's cheeks and chin. The mask structure 1, therefore, can be conveniently put on and securely worn.

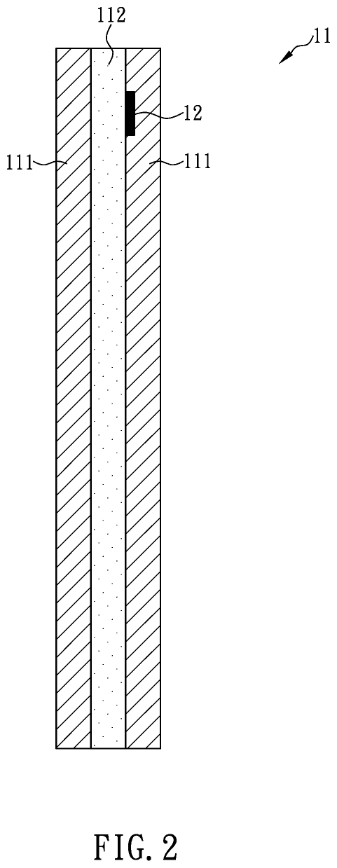

As shown in FIG. 1 and FIG. 2, the mask body 11 includes at least one air-permeable layer 111. The air-permeable layer 111 may be made of a textile (e.g., non-woven fabric, cotton cloth, etc.) and can filter out airborne particulate matter. In certain embodiments, the mask body 11 includes two air-permeable layers 111 and a filter layer 112 (see FIG. 2). The two air-permeable layers 111 may be, but are not limited to, non-woven fabric, as long as they allow passage of air. The two air-permeable layers 111 can even differ from each other in material and structural details. For example, the outer air-permeable layer 111 (i.e., the air-permeable layer 111 exposed when the mask structure 1 is worn) can have a water-repellent structure to block out substances such as flying saliva, water drops, dust, and blood, and the inner air-permeable layer 111 (i.e., the air-permeable layer 111 adjacent to the wearer's face) can have a water-absorbent and skin-friendly structure for adsorbing the wearer's sweat, oil on the face, and saliva.

With continued reference to FIG. 1 and FIG. 2, the filter layer 112 has two opposite sides whose respective surfaces are each covered by the surface of the corresponding side of one of the air-permeable layers 111. The filter layer 112 further has air passage holes, whose diameters are typically smaller than those of the air passage holes of the two air-permeable layers 111 in order for the filter layer 112 to filter out airborne particulate matter. The filter layer 112 can be melt-blown non-woven fabric, whose melt-blown mesh structure provides electrostatic adsorption and can remove more than 90% of 5-μm particles (which are approximately the sizes of cells and bacteria). In certain embodiments, the filter layer 112 can be, but is not limited to, a polymeric air-permeable film. Depending on actual product requirements, the filter layer 112 can also be a mesh layer containing activated carbon in order to adsorb volatile organic compounds (VOCs) and odorous molecules in the air, and the diameters of the air passage holes of the filter layer 112 can be equivalent to those of the air passage holes of the two air-permeable layers 111. Moreover, while FIG. 2 shows a three-layer configuration, in certain embodiments, a certain one or certain ones of the layers may be omitted or another layer or other layers be added to meet product requirements, and as long as a mask body includes at least one air-permeable layer 111 for filtering out airborne particulate matter, it falls within the scope of the mask body 11 defined in the present disclosure.

In certain embodiments, the scent releasing element 122 can be an aroma compound (e.g., a spice, perfume, fragrance essence, or essential oil) itself or a medicinal compound. Aroma compounds can be esters (e.g., geranyl acetate, ethyl butyrate, etc.), linear terpenes (e.g., myrcene, nerol, citronella1, etc.), cyclic terpenes (e.g., limonene, menthol, thujone, etc.), aromatics (e.g., cinnamaldehyde, anisole, etc.), and amines (e.g., indole). These aroma compounds can produce molecules of different scents respectively, such as a fruity scent, a floral scent, a woody scent, a pepperminty scent, etc. Once entered the nostrils, an aroma compound can react with the olfactory cells in the nasal cavity such that bioelectric waves are generated and propagate to the brain through the corresponding nerves. The inhaled fragrances of aroma compounds can loosen up stiff muscles (a physical effect) and produce a nerve-calming, stress-relieving, and relaxing effect (a psychological effect). For example, peppermint oil is refreshing and invigorating and can improve concentration, relieve headaches, and reduce nervousness; tea tree oil has a deodorizing effect and can purify the air; rosemary oil is invigorating and can intensify the pulsation of the heart, stimulate the brain and the central nervous system, and alleviate headaches, migraines, and neuralgia; and lavender oil can soothe one's anxiety and improve sleep quality. It should be pointed out that aroma compounds for use in the present disclosure are not limited to the essential oils mentioned above, and as long as a scent releasing element can produce scent molecules that can react with the olfactory cells in the nasal cavity, it falls within the scope of the scent releasing element 122 defined in the present disclosure.

In certain embodiments, referring to FIG. 3, the scent releasing element 122 can be combined with the main body 121 to meet actual product requirements. For example, the main body 121 can be a plastic material (e.g., ethylene-vinyl acetate (EVA)), and the scent releasing element 122 (e.g., a perfume, fragrance essence, essential oil, spice, etc.) is added to the plastic material (e.g., EVA granules). During the adding process, the plastic material may be in a granular state, and the scent releasing element 122 in a liquid state. After that, the scent releasing element 122 and the plastic material are melted at a high temperature to form a liquid mixture. The mixture is then extruded or goes through a film blowing process to form a substantially 1.0 mm-thick and 4.0 mm-wide nose bridge strip 12. Since the scent releasing element 122 is mixed in the plastic material and hence tightly combined with the main body 121, the main body 121 can stably and persistently retain the scent molecules produced by the scent releasing element 122. In addition, the nose bridge strip 12 has such advantages as a simple manufacturing process, low cost, and ease of production.

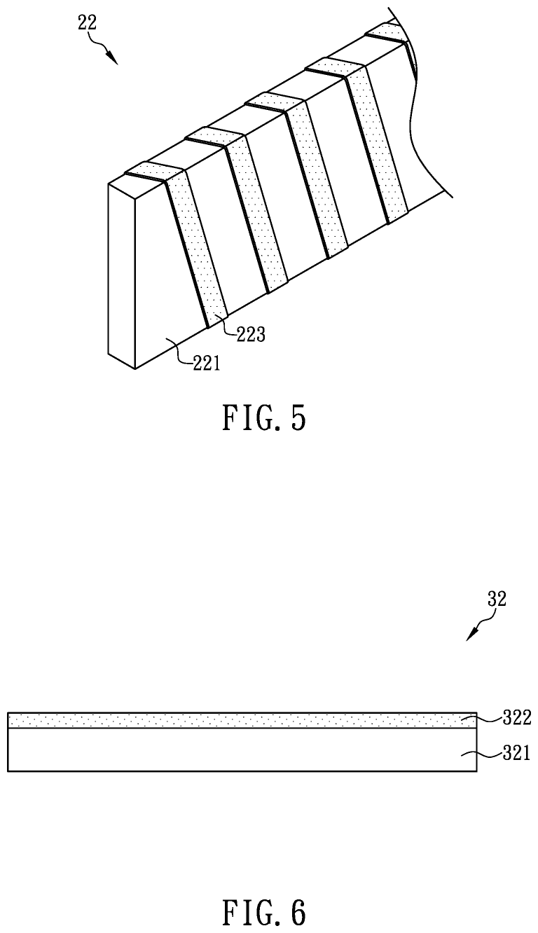

In certain embodiments, referring to FIG. 4 and FIG. 5, a nose bridge strip 22 includes a main body 221 and a scent releasing element 222 that are independent of each other. The main body 221 can be made of a plastic material or a metal material (e.g., iron, an aluminum alloy, etc.). The scent releasing element 222 can be in a shape of a film and then fixed on a side surface of the main body 221. For example, one side surface of a plastic film 223 can be fixedly coated with a scent releasing layer 224 (e.g., of an aroma compound or medicinal compound), and once the scent releasing layer 224 is dry or solidified, an adhesive 225 is applied over the opposite side surface of the plastic film 223. After that, the plastic film 223 may be fixed on the side surface of the main body 221 by winding, weaving, or adhesive bonding. For example, the scent releasing element 222 can be directly adhered to, and lie flat on, the surface of one side surface of the main body 221. Or, the scent releasing element 222 can be wound on the side surface of the main body 221, with each two adjacent turns of the plastic film 223 spaced apart from each other (see FIG. 5) or overlapping each other. In either case, the nose bridge strip 22 has a scent releasing mechanism implemented by the scent releasing element 222 producing scent molecules.

While the scent releasing layer 224 in FIG. 4 can be on an outer side surface of the plastic film 223, the present disclosure has no limitation on the location of the scent releasing layer 224 in relation to the plastic film 223. The scent releasing layer 224 in certain embodiments of the present disclosure can be located on the inner side surface of the plastic film 223, as long as the scent molecules produced by the scent releasing layer 224 can pass through the plastic layer 223. In certain embodiments, the scent releasing element 222 may dispense with the adhesive 225 and be directly wound around the main body 221 before being placed into the mask body 11 (e.g., between one of the air-permeable layers 111 and the filter layer 112) along with the main body 221 in order not to separate from the mask body 11 but to release scent molecules continuously in the mask body 11. In certain embodiments, the scent releasing element 222 itself can be a film made of an aroma compound or medicinal compound. For example, an aroma compound (or medicinal compound) is mixed with an adhesive and allowed to dry or solidify in order to form a film, which in turn is fixed on a side surface of the main body 221 by winding, weaving, or adhesive bonding such that each two adjacent turns of the film are spaced apart from or overlap each other on the side surface of the main body 221.

In certain embodiments, referring to FIG. 6, a main body 321 can be of a plastic material or a metal material, and a side surface of the main body 321 can be directly and fixedly coated with a scent releasing element 322 in order to form a nose bridge strip 32. For example, an aroma compound (or medicinal compound) is mixed with an adhesive, and the mixture is applied over the side surface of the main body 321. In certain embodiments, the main body 321 can be dipped into the mixture and allowed to dry or solidify such that the scent releasing element 322 is attached to the side surface of the main body 321. The method of providing the scent releasing element 322, however, is not limited to those described above. In certain embodiments, the adhesive may be dispensed with, and the aroma compound (or medicinal compound) is directly applied, in a liquid state, over the side surface of the main body 321, or the main body 321 is dipped into the liquid aroma compound (or medicinal compound), allowing the scent releasing element 322 to seep into the pores of the main body 321 or cover the side surface of the main body 321 in order to produce scent molecules.

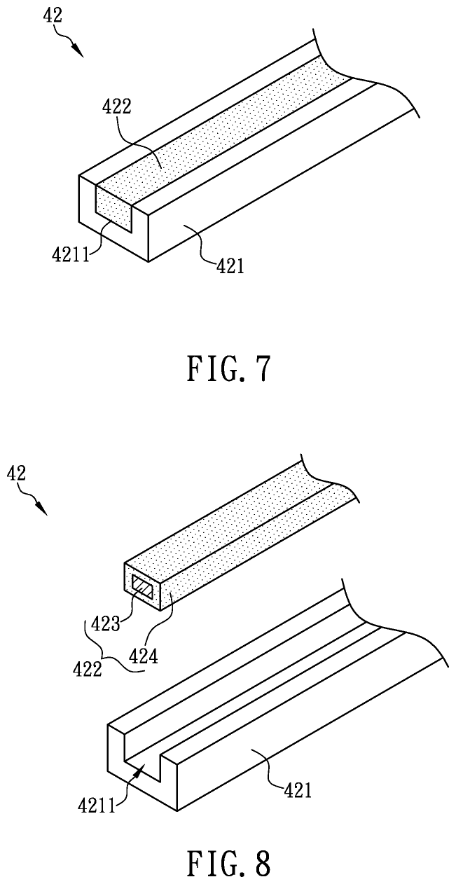

In certain embodiments, referring to FIG. 7 and FIG. 8, the main body 421 of a nose bridge strip 42 can be of a plastic material or a metal material, and a groove 4211 is provided on one side surface of the main body 421 so that the scent releasing element 422 can be securely received in the groove 4211. For example, the film-like scent releasing element 222 in certain embodiments may be adhesively attached to the wall of the groove 4211, or the liquid aroma compound (or medicinal compound) in certain embodiments may be injected into the groove 4211 in order to fixedly coat the wall of the groove 4211 with the scent releasing element 322, or the scent releasing element 422 can be made as a strip-like object and fixed in the groove 4211 (e.g., at least one side surface of a plastic strip 423 can be fixedly coated with a scent releasing layer 424 (e.g., of an aroma compound of medicinal compound) and then received in the groove 4211). Once the mask structure 1 is properly put on a user's face, the nose bridge strip 42 corresponds in position to the bridge of the user's nose and can be pressed and deformed compliantly against the bridge. The nose bridge strip 42 will release the scent molecules produced by the scent releasing element 422, and the scent molecules will pass sequentially through the nasal mucosa, the olfactory receptors, the olfactory bulbs, the olfactory tracts, and the limbic system to the hypothalamus, allowing the wearer of the mask structure 1 to smell a pleasant scent and feel comfortable while breathing. Thus, the nose bridge strip with the scent releasing mechanism and the mask structure having the same according to the present disclosure can help prevent physical and mental diseases and contributes to the maintenance of health.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A nose bridge strip having a scent releasing mechanism, comprising:
a main body in a shape of a strip and configured to be fixed on or in a mask body at a location corresponding to a nose bridge on a human face when the mask body is worn on the human face and be pressed and deformed compliantly against the nose bridge; and
at least one scent releasing element configured to be combined with the main body and produce scent molecules capable of reacting with olfactory cells in a nasal cavity.

2. The nose bridge strip according to claim 1, wherein the main body is made of a plastic material, and the scent releasing element solidified from a liquid state is mixed with the main body to form the nose bridge strip.

3. The nose bridge strip according to claim 1, wherein the scent releasing element is in a shape of a film and fixed on a side surface of the main body in a winding or weaving configuration or by adhesive bonding.

4. The nose bridge strip according to claim 1, wherein the scent releasing element is fixed on a side surface of the main body by directly applying the scent releasing element to the side surface or dipping the main body in a mixture including the scent releasing element.

5. The nose bridge strip according to claim 1, wherein one side surface of the main body is provided with a groove for receiving and fixing the scent releasing element therein.

6. The nose bridge strip according to claim 1, wherein the main body is made of a plastic material or a metal material.

7. The nose bridge strip according to claim 1, wherein the scent releasing element includes an aroma compound or medicinal compound.

8. A mask structure having a scent releasing mechanism, comprising:
a mask body including at least one air-permeable layer;
ear straps provided respectively at the left and right sides of the mask body and configured to be wrapped around left and right ears of a user and bring an inner-side surface of the mask body close to a face of the user;
a main body in a shape of a strip and configured to be fixed on or in a mask body at a location corresponding to a nose bridge on the face when the mask body is worn on the face and be pressed and deformed compliantly against the nose bridge; and
at least one scent releasing element configured to be combined with the main body and produce scent molecules capable of reacting with olfactory cells in a nasal cavity.

9. The mask structure according to claim 8, wherein the main body is made of a plastic material, and the scent releasing element solidified from a liquid state is mixed with the main body to form the nose bridge strip.

10. The mask structure according to claim 8, wherein the scent releasing element is in a shape of a film and fixed on a side surface of the main body in a winding or weaving configuration or by adhesive bonding.

11. The mask structure according to claim 8, wherein the scent releasing element is fixed on a side surface of the main body by directly applying the scent releasing element to the side surface or dipping the main body in a mixture including the scent releasing element.

12. The mask structure according to claim 8, wherein one side surface of the main body is provided with a groove for receiving and fixing the scent releasing element therein.

13. The mask structure according to claim 8, wherein the main body is made of a plastic material or a metal material.

14. The mask structure according to claim 8, wherein the scent releasing element includes an aroma compound or medicinal compound.
